(19) United States
(12) Patent Application Publication
(10) Pub. No.: US 2019/0100553 A1
(43) Pub. Date: Apr. 4, 2019

(54) DIRECT ACTIVITY ASSAYS AND COMPOSITIONS FOR NUCLEOTIDE POOL SANITIZING ENZYMES

(71) Applicant: The Board of Trustees of the Leland Stanford Junior University, Stanford, CA (US)

(72) Inventors: Eric T. Kool, Stanford, CA (US); Debin Ji, Palo Alto, CA (US)

(73) Assignee: The Board of Trustees of the Leland Stanford Junior University, Stanford, CA (US)

(21) Appl. No.: 16/086,488

(22) PCT Filed: Mar. 23, 2017
(86) PCT No.: PCT/US2017/023856

§ 371 Date: Sep. 19, 2018

Related U.S. Application Data
(60) Provisional application No. 62/321,368, filed on Apr. 12, 2016.

(51) Int. Cl. C07H 19/207; C07H 19/24; G01N 33/52; G01N 33/58

(52) U.S. Cl. CPC C07H 19/207; C07H 19/24; G01N 2500/10; G01N 33/582; G01N 33/52

(57) ABSTRACT

Compositions and methods are provided for detecting activity of nucleotide pool repair enzymes. In the methods of the invention, a sample suspected of having nucleotide pool repair enzyme activity is combined with a detection compound provided herein, which detection compound comprises (i) a substrate for the enzyme of interest, (ii) a polyphosphate linker; and (iii) a detection moiety that is active when released by the enzyme of interest cleaving the linker.

### CROSS REFERENCE

This application claims benefit of PCT Application No. PCT/US2017/023856, filed Mar. 23, 2017, which claims benefit of U.S. Provisional Patent Application No. 62/321,368, filed Apr. 12, 2016, which applications are incorporated herein by reference in their entirety.

### FEDERALLY SPONSORED RESEARCH AND DEVELOPMENT

This invention was made with Government support under contract GM068122 awarded by the National Institutes of Health. The Government has certain rights in the invention.

### BACKGROUND OF THE INVENTION

Cellular reactive oxygen species (ROS) can damage DNA in multiple ways. This includes not only the direct damage to the heterocyclic bases and deoxyribose sugars of DNA itself, but also to the cellular nucleotides that are used to synthesize new DNA during cell division. Cells require accurate replication of genetic information for successful replication. In addition to mechanisms such as polymerase proof-reading functions and mismatch repair, cells also maintain systems for sanitizing the nucleotide pool. Nucleotides that are damaged, e.g. by oxidation, deamination, etc. may be incorporated by polymerases into DNA or RNA, leading to potentially harmful mutations and DNA damage. Pathways for “sanitizing” the nucleotide pool include enzymes that hydrolyze NTPs containing modified bases, thereby preventing the modified bases from incorporation. Typically such enzymes have a high-specificity recognition pocket for the modified base. Several of these enzymes catalyze the cleavage of the phosphoanhydride linkage within the phosphate chain of the NTPs, resulting in a nucleoside monophosphate and inorganic pyrophosphate.

The most abundant forms of oxidative damage in the cellular deoxynucleotide pool are 8-oxo-dGTP and 2-hydroxy dATP. 8-Oxoguanine (8-oxoG) is one of the major oxidized bases in DNA or the nucleotide pool and is highly mutagenic because it can pair with adenine as well as cytosine in DNA, leading to mutations. The most abundant forms of deamination damage in the nucleotide pool are dUTP, dITP and ITP, and dXTP and XTP.

Important enzymes for nucleotide pool repair include Nudix enzymes, characterized by the conserved Nudix domain GX₅EX₇REUXEEXGU (where U represents hydrophobic residues such as Ile, Val and Leu). This large family of enzymes includes members, such as MTH1, with a key role in maintaining both DNA replication and translational fidelity by performing pyrophosphorolysis on oxidized nucleotides 8-oxo-dGTP and 8-oxo GTP. Human MTH1 binds 8-oxo-dGTP and hydrolyzes its triphosphate moiety between the α and β phosphates, producing inactive 8-oxo-dGMP and pyrophosphate.

While MTH1 activity is important for suppressing mutations in normal cells, it is not essential for cell viability (Tsuzuki et al. (2001) PNAS 98:11456-11461). However, cancer cells can be highly dependent on MTH1 to maintain their rapid growth (Giribaldi et al. (2015) Oncotarget 6:11519-11529). For example, various selective inhibitors of MTH1 induce an increase in DNA single-strand breaks, activated DNA repair in human colon carcinoma cells and effectively suppressed tumor growth in animal models (see Gad et al. (2014) Nature 508:215). Many tumors are driven by mutations in the RAS proto-oncogenes that increase reactive oxygen species, resulting in damage such as 8-oxo-dG (Rai et al. (2011) Oncogene 30:1489-1496). Thus RAS-dependent tumor cells often express high MTH1 levels to counteract the toxicity of elevated ROS in these rapidly growing cells (Patel et al. (2015) Oncogene 34:2586-2596; Coskun et al. (2015) DNA Repair 33:101-110). Such studies suggest the promise of targeting MTH1 as a new therapeutic approach for multiple RAS-driven cancers, which may include without limitation melanoma and colorectal cancer (Huber et al. (2014) Nature 508:222-227).

Other nucleotide pool repair enzymes include dUTPases, which catalyze the pyrophosphate cleavage of dUTP into dUMP and pyrophosphate. dUTPase deficiency leads to the appearance of highly uracil-substituted DNA. The enzyme is developmentally regulated in higher organisms, where the highest activity is found in the non-differentiated, actively proliferating cells. Based on the significant role of the enzyme in DNA metabolism, it has been proposed as a target for anticancer drugs and potentially antiviral drugs.

Defects in purine nucleotide metabolism can result in the incorporation of hypoxanthine and xanthine into DNA and RNA. The deaminated purine NTPs, ITP and XTP, and their reduced derivatives, dITP and dXTP, are degraded by ITPase. The incorporation of dITP or dXTP into DNA is not mutagenic in itself, but as the DNA repair machinery attempts its removal this can lead to DNA strand breaks. The ITPA variant in humans is associated with drug sensitivity, as well as with elevated level of DNA strand breaks.

Inhibition of nucleotide pool repair enzymes is quite promising from the clinical oncology perspective. In developing targeted therapies for these enzymes, it will be important to measure enzyme activity in tumor specimens, for patient selection, to monitor responses to therapy, and to evaluate any resistance that arises. Moreover, simple high-throughput methods for quantifying their activity is valuable for identifying new inhibitors and improving existing ones. Despite the growing clinical significance of this enzyme, current methods to measure these activities are laborious and indirect, and have low sensitivity.

For example, the most common methods of measuring MTH1 activity include HPLC measurement of nucleotide cleavage to 8-oxo-dGMP, (see Takagi et al. (2012) J. Biol. Chem. 287:21541-21549) which is limited by the relatively low UV absorbance of the nucleotide, and a colorimetric assay involving the use of pyrophosphatase to degrade the pyrophosphate byproduct to inorganic phosphate, which is then detected after complexation with molybdate and malachite green dye (Feng et al. (2011) Anal. Biochem. 409:144-149). Because of the low sensitivity and laborious nature of these methods, most studies do not measure MTH1 activity, but instead evaluate indirectly related metrics such as mRNA quantities or protein levels which may not correlate linearly with cellular enzyme activity. A recent study, citing the need for measuring MTH1 for clinical development, reported a proteomics method involving isotope labeling, protease digestion, HPLC and tandem mass spectrometry, (Coskun et al., supra.) which due to its complexity may be difficult to develop for routine clinical use. In addition, for most applications, it would be preferable to measure MTH1 activity rather than protein quantities, since the enzyme may have variable activity due to single nucleotide variations (Goto et al. (2008) J. Genet. 87:181-186).

The present invention provides compositions and methods for simple, sensitive and fast assays for activity of nucleotide pool repair enzymes.

### SUMMARY OF THE INVENTION

Compositions and methods are provided for detecting activity of nucleotide pool repair enzymes. In the methods of the invention, a sample suspected of having nucleotide pool repair enzyme activity is combined with a detection compound provided herein, which detection compound comprises (i) a substrate for the enzyme of interest, (ii) a polyphosphate linker; and (iii) a detection moiety that is active when released by the enzyme of interest cleaving the linker. The released detection moiety may be directly or indirectly detected by any suitable means, e.g. quantitating fluorescence, fluorescence, etc. Samples of interest include biological samples, e.g. biopsy tissue, cell lysate, in vitro cell culture, blood sample, etc., or may be a sample from compound screening, etc.

In some embodiment of the invention, detection compounds are provided, which have the general formula I:

N-L-D I

where N is a modified nucleoside including, without limitation, nucleosides comprising 8-oxoguanine, xanthine, hypoxanthine, 2-hydroxy adenine, uracil, 6-methylpurine, etc. and a ribose or deoxyribose sugar. Where the nucleobase is uracil, the sugar is deoxyribose. L is a polyphosphate linker of from about 3 to 6 or more phosphates in length, e.g. having 3, 4, or 5 phosphates. D is a detectable moiety. In some embodiments D is adenosine, which upon cleavage by the enzyme of interest is released as ATP. In other embodiments D is a fluorophore that is quenched when linked to the modified nucleoside, but detectable when released.

Enzymes of interest for analysis by the methods of the invention can be derived from any organism, as nucleotide pool repair is a common feature of living cells. In some embodiments the enzyme of interest is a mammalian enzyme, particularly a human enzyme. Of particular interest are phosphohydrolase enzymes with specificity for modified bases; which include, without limitation, MTH1, also known as NUDT1, which acts on 8-oxo-GTP or dGTP; dUTPase (DUT), which acts on dUTP; ITPase (ITPA), which acts on XTP, dXTP, ITP, dITP; etc.

In some embodiments, methods are provided for quantitating activity of one or more nucleotide pool repair enzymes in a sample, e.g. a sample containing cells, tissues, or a lysate thereof; a sample containing the enzyme of interest and candidate inhibitor; a sample suspected of containing the enzyme of interest, and the like. The sample is combined with a detection compound of the invention under conditions permissive for activity of the enzyme of interest. After a period of time sufficient for the reaction to proceed, the sample is analyzed for the presence of released detectable moiety. The level of activity may be compared to a control, e.g. a known standard, a control cell population, etc. Fluorescent moieties may be directly detected. ATP can be detected by various methods known in the art, including luciferase activity, activation of fluorophores, and the like. When ATP comprises the detectable moiety, cellular samples may be ATP-depleted prior to analysis.

Sample comprising cells, tissues, or lysates thereof may be cancer cells or suspected cancer cells, including without limitation cancer cells associated with activity of a ras oncogene. Cancers of interest include melanoma, lymphoma, carcinoma, e.g. adenocarcinoma, squamous cell carcinoma, sarcoma, glioblastoma, leukemia, etc., which may be lung cancer, colorectal cancer, breast cancer, pancreatic cancer, colorectal cancer, prostate cancer, ovarian cancer, head and neck cancer, etc. Cells, including cancer cells, can be provided from in vitro cultures, biopsies, scraped cells, blood samples, etc.

In some embodiments a cancer patient is selected for treatment when a cancer cell population is found to have a predetermined level of nucleotide pool repair activity, e.g. an increase of greater than about 25%, greater than about 50%, greater than about 75%, greater than 2-fold, greater than 3-fold, greater than 5-fold, greater than 10-fold or more relative to a normal control or standard. Similarly, for some uses, patients may be selected for low activity of a specific enzyme. Treatment may include, for example, administration of an inhibitor of the nucleotide pool repair enzyme, alone or in combination with immunotherapy, chemotherapy, radiation therapy, or surgery; selection of a chemotherapeutic or immunotherapeutic agent; and the like. In some embodiments, monitoring of nucleotide pool repair activity in performed during the course of therapy. In some embodiments, the analytic methods of the invention are used to select an individual for treatment, including selection for inclusion in clinical trials.

In another embodiment there is provided a method of selecting dosage of a cancer treatment, such as chemotherapy and/or radiotherapy, for a subject, the method comprising determining a level of activity of a nucleotide pool repair enzyme in a tissue or sample thereof, and, according to the level of activity, selecting dosage of the treatment for the subject.

The efficacy of therapy in a patient with cancer or other condition associated with upregulation of nucleotide pool repair enzyme activity can be assessed by detecting activity of nucleotide pool repair enzymes by the methods of the invention.

Various formats may be used in the analysis of activity. In some embodiments, a patient sample is obtained prior to treatment, as a control, and compared to samples from the same patient following treatment. In other embodiments, the activity is assessed over long periods of time to monitor patient response or status.

In some embodiments a method is provided for screening drug candidates for modulation of nucleotide pool repair activity. A drug candidate, which may inhibit or enhance enzyme activity, is combined with a detection compound of the invention. A nucleotide pool repair enzyme is added to the reaction mix, and the release of the detectable moiety is monitored. The level of activity in the presence of the drug candidate is compared to a control to determine the inhibition or potentiation of activity. Assays may be performed in a high throughput system.

Kits are provided, which comprise at least one or more detection compound(s) of the invention. A kit may comprise 1, 2, 3 or more different detection compounds. The detection compound(s) may be provided in a unit dose, e.g. for clinical use, or in a multi-unit form suitable for dilution, e.g. for high throughput screening assays. Optionally reaction buffers, control samples, and/or additional reagents such as luciferase are provided.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. The chimeric OGAL probe combines 8-oxo-dGTP (damaged base shown in blue) and ATP. The MTH1 repair enzyme cleaves the probe, releasing ATP, which can then trigger luminescence signals via luciferase and luciferin.

FIG. 2A-2C. OGAL acts as an efficient substrate of MTH1, generating luciferase signals. (FIG. 2A) Michaelis-Menten plot of OGAL as a substrate. ^{a}Values from previous published work. (FIG. 2B) Plot of luciferase signals as a function of time of MTH1 reaction with 40 μM OGAL (two-tube reaction). (FIG. 2C) Time course of signals in single-tube reaction (control=no MTH1). See text for conditions; data in (FIG. 2A, 2B) are averaged from three experiments.

FIG. 3A-3B. Use of OGAL probe to quantify MTH1 and evaluate inhibitors. (FIG. 3A) Sensitivity of the OGAL probe as measured by dilutions of the enzyme. Limit of detection is 0.5 nM enzyme using the two-tube assay. (FIG. 3B) Titration curves of MTH1 activity in vitro with two inhibitors, measured with two-tube assay. (S)-crizotinib was previously reported as an inhibitor, while NVP-AEW541 was discovered in this work.

FIG. 4A-4B. Employing the OGAL probe to measure MTH1 activities in cell lysates. (FIG. 4A) Time course plot of luciferase signal in U2OS lysate in the absence (−) and presence (+) of inhibitor (S)-crizotinib; buffer control lacks cells. (FIG. 4B) Plot of MTH1 activity in varied tumor cell lines. Lysates were depleted of ATP before measurement as described in the Supporting Information. Error bars show standard deviations from three measurements.

FIG. 5. HPLC analysis showing cleavage of OGAL probe by MTH1 into 8-oxo-dGMP and ATP (these two species coelute). Product peaks were determined by co-injection (not shown). Reaction conditions: 100 μM OGAL nucleotides and 40 nM MTH1 reacted in buffer (50 mM NaCl, 10 mM Tris-HCl (pH 7.9), 10 mM MgCl₂, 1 mM DTT). After 2 h incubation at 30° C., the enzyme was removed by CHCl₃ extraction. The product was analyzed by HPLC using C18 column with a gradient of acetonitrile and 50 mM triethylammonium acetate buffer (pH 7).

FIG. 6. MS data showing molecular masses of MTH1-mediated OGAL cleavage products 8-oxo-dGMP and ATP. The HPLC peak containing ATP and 8-oxo-dGMP were collected and concentrate. Then desalt by HPLC to remove the TEAA buffer.

FIG. 7A-7B. (FIG. 7A) OGAL probe reaction with luciferase shows low background, (note small and rapid luminescence signals as compared with an equimolar quantity of ATP. The background signals (red, 2-12 min) likely arise from very small contamination of OGAL with ATP. (FIG. 7B) ATP vs. OGAL background signal quantities by sum all signals over 60 min. Error bars show standard deviation over 3 measurements.

FIG. 8. Testing origin of drop in luminescence signals in one-tube reaction with OGAL probe. Second aliquot of luciferase reaction buffer with luciferase was added after 5 h. The new signal confirms that MTH1 enzyme remains active, confirming that the end of signal in the first instance was due to the deactivation of luciferase over time.

FIG. 9A-9B. Inhibitor screening with OGAL/luciferase assay. (FIG. 9A) Plots of luminescence in one-tube OGAL probe reactions at 5 μM inhibitor concentrations. (FIG. 9B) Titration of activities of 5 potential inhibitors at four concentrations. Data show luminescence intensities at 10 min. Inhibitor 1 is NVP-AEW541 (see titration plot in FIG. 3, panel B).

FIG. 10. ATP depletion of cell lysates by washing with centrifugal filter columns. ATP levels (measured by luciferase kit) in original cell lysate and each times of filtrate. To measure ATP levels, 5 μL of the original U2OS cell lysate or 5 μL of filtrate from spin column filtration was added to 95 μL luciferase reaction buffer and the luminescence signals at 5 min were recorded. Columns 1-5 show signals after each of 5 successive washes.

FIG. 11. Employing the OGAL probe to measure MTH1 activities in tissue lysates. 30 μg (total protein) tissue lysate was added to 20 μL MTH1 reaction buffer with or without 20 μM (S)-crizotinib inhibitor. The MTH1 reaction buffer contained 50 mM NaCl, 10 mM Tris-HCl (pH 7.9), 10 mM MgCl₂, 1 mM Na₃VO₄, 1 mM DTT, 40 μM OGAL nucleotide and 0 μM or 20 μM (S)-crizotinib. After 1 h incubation at 30° C., 5 μL of this reaction solution was added to 95 μL luciferase reaction solution in a 96 well plate, and the luminescence signal at 5 min was recorded. To calculate MTH1-specific signals, background level with inhibitor for each lysate was subtracted from that without inhibitor. To obtain absolute enzyme levels, these data were fit to the standard dilution plot of signal from pure MTH1 (see FIG. 3, panel A). All data were the averages of nine replicates. (**p=0.01)

FIG. 12. ¹H-NMR and ³¹P-NMR spectra (D₂O) of OGAL chimeric nucleotide.

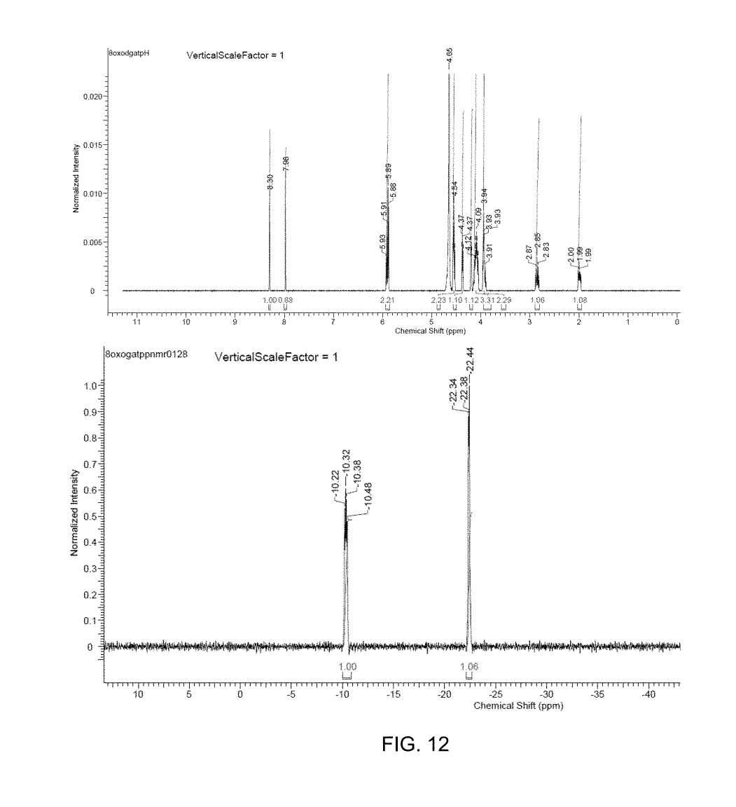

FIG. 13A-13D. Measurement of ITPA activity. (FIG. 13A) Structure of DIAL probe. (FIG. 13B) Activity of ITPA on the DIAL probe. (FIG. 13C) Michaelis-Menton analysis of ITPA using DIAL probe. (FIG. 13D) Measuring ITPA activity in yeast extracts with DIAL probe.

FIG. 14A-14C. Measurement of dUTPase activity. (FIG. 14A) Structure of DUAL probe. (FIG. 14B) Activity of dUTPase on the DUAL probe. (FIG. 14C) Michaelis-Menton analysis of dUTPase using DUAL probe.

### DETAILED DESCRIPTION OF THE INVENTION

The present invention provides compositions, methods and kits useful for the luminescence or fluorescence detection of the activity of enzymes that cleave between phosphates of a nucleoside triphosphate for nucleotide pool repair.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing those components that are described in the publications that might be used in connection with the presently described invention.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.); Molecular Probes (Eugene, Oreg.); Applied Biosystems, Inc. (Foster City, Calif.); and Glen Research (Sterling, Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

Unless otherwise apparent from the context, all elements, steps or features of the invention can be used in any combination with other elements, steps or features.

The terms “subject,” “individual,” and “patient” are used interchangeably herein and may refer to a mammal being assessed for treatment and/or being treated. In an embodiment, the mammal is a human. The terms “subject,” “individual,” and “patient” may encompass, without limitation, individuals having cancer or suspected of having cancer. Subjects may be human, but also include other mammals, particularly those mammals useful as laboratory models for human disease, e.g. mouse, rat, etc. Also included are mammals such as domestic and other species of canines, felines, and the like. For other purposes, nucleotide pool repair enzymes from diverse organisms, including e.g. bacteria, protozoa, fungi, plants, etc. can be analyzed for activity using the compositions and methods of the invention.

The terms “cancer,” “neoplasm,” and “tumor” are used interchangeably herein and may refer to cells that exhibit autonomous, unregulated growth, such that they exhibit an aberrant growth phenotype characterized by a significant loss of control over cell proliferation. Cells of interest for detection, analysis, or treatment in the present application may include, but are not limited to, precancerous (e.g., benign), malignant, pre-metastatic, metastatic, and non-metastatic cells. Cancers of virtually every tissue are known. The phrase “cancer burden” may refer to the quantum of cancer cells or cancer volume in a subject. Reducing cancer burden accordingly may refer to reducing the number of cancer cells or the cancer volume in a subject. The term “cancer cell” as used herein may refer to any cell that is a cancer cell or is derived from a cancer cell, e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include, but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The “pathology” of cancer may include, but it not limited to, all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

“Measuring” or “measurement” in the context of the present teachings may refer to determining the presence, absence, quantity, amount, or effective amount of a substance in a clinical or subject-derived sample, including the presence, absence, or concentration levels of such substances, and/or evaluating the values or categorization of a subject's clinical parameters based on a control.

The term “diagnosis” may refer to the identification of a molecular or pathological state, disease or condition, such as the identification of a molecular subtype of melanoma, colorectal cancer, breast cancer, prostate cancer, or other type of cancer.

The term “prognosis” may refer to the prediction of the likelihood of cancer-attributable death or progression, including recurrence, metastatic spread, and drug resistance. The term “prediction” may refer to the act of foretelling or estimating, based on observation, experience, or scientific reasoning.

The terms “treatment,” “treating,” and the like, may refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, e.g., arresting its development; and (c) relieving the disease, e.g., causing regression of the disease.

As used herein, “methods known to one of ordinary skill in the art” may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The compounds of the invention may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “reagent mix”, as used herein, refers to a combination of reagents, that are interspersed and not in any particular order. A reagent mix is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct.

ATP detection reagent(s). Many reagents and assays are known in the art for use in detecting the presence of ATP. For the purposes of the present invention, these reagents are used to detect ATP released by enzyme cleavage of the linker, and thus provide a qualitative or quantitative assessment for the activity of the enzyme. ATP detection reagents include without limitation luciferase bioluminescence assays (see, for example, J Appl Biochem 3, 473 (1981); Fraga (2008) Photochemical & Photobiological Sciences 7(2):146-158; Bell et al. (2007) Methods Cell Biol. 80:341-352), fluorescent dyes, target-responsive aptasensors, glass bead microarray, GO-nS nanocomplex platform, and the like. In certain embodiments the assay utilizes detection of light produced by luciferin and luciferase.

Exemplary fluorescent dyes are described, for example in Jose et al. (2007) Org. Lett. 9:1979-1982; Lee et al. (2004) Angew. Chem. Int. Ed. 43:4777-4780; Sancenon et al. (2001) Angew. Chem. Int. Ed. 40:2640-2643; Mizukami et al. (2002) JACS 124:3920-3925; Schneider et al. (2000) JACS 122:542-543; Ojida et al. (2006) Angew. Chem. Int. Ed. 45:5518-5521; Li et al. (2005) Angew. Chem. Int. Ed. 44:6371-6374, each of which is herein specifically incorporated by reference.

Target responsive aptamers are described, for example, by Li & Ho (2008) JACS 130:2380-2381; Li & Lu (2006) Angew. Chem. Int. Ed. 45:90-94; Zayats (2006) JACS 128:13666-13667. Glass bead microarrays are described by McClesky et al. (2003) JACS 125:1114-1115. A GO-nS nanocomplex platform is described by Wang et al. (2013) Anal. Chem. 85:6775-6782. Each of these references is herein specifically incorporated by reference.

The term “luciferase” refers to an adenosine triphosphate (ATP) hydrolase that catalyzes the hydrolysis of ATP into constituent adenosine monophosphate (AMP) and pyrophosphate (PPi) along with the release of light. A luciferase has an activity described as EC 1.13.12.7, according to IUBMB enzyme nomenclature. A luciferase of interest is Photinus luciferin 4-monooxygenase (ATP-hydrolyzing).

Luciferin is a common bioluminescent reporter used for in vitro assays in combination with luciferase. This water soluble substrate for luciferase enzymes (e.g. Photinus pyralis, Cypridina, Gaussia, Renilla, etc. utilizes ATP and Mg²⁺ as co-factors to emit a characteristic yellow-green emission in the presence of oxygen. Many reagents and kits are commercially available for this purpose. When luciferin and luciferase are combined in a reaction mixture comprising ATP, there is an immediate flash of light that reaches peak intensity within 0.3-0.5 seconds. The light then begins to decay rapidly with a half-life around 0.5-1.0 min. The optional addition of Coenzyme A to the reaction mixture prevents the fast reaction decay, extending the half-life of the reaction from 2-5 minutes. Luciferin analogs known to be substrates of luciferase enzyme are also contemplated.

Fluorophores. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′, 5′,7′,1,4-hexachlorofluorescein (HEX), and 2′,7′-dimethoxy-4′,5′-dichloro-6-carboxyfluorescein (JOE); cyanine dyes, e.g. Cy3, CYS, Cy5.5, etc.; dansyl derivatives; 6-carboxytetramethylrhodamine (TAMRA), BODI PY fluorophores, tetrapropano-6-carboxyrhodamine (ROX), ALEXA dyes, Oregon Green, pyrene, perylene, benzopyrene, oxoperylene, rubrene, perylene bisimide, styrene, anthracene, tetracene, pentacene, fluorine, phenanthrene, stilbene, dimethylaminostilbene, quinacridone, fluorophenyl-dimethyl-BODIPY, bis-fluorophenyl-BODIPY, acridine, terrylene, sexiphenyl, porphyrin, phenylporphyrin, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl (multiple isomers possible), and ter-naphthyl, tricyclic cytosine analogs, 1,3-diaza-2-oxophenothiazine (tC), and the like.

Fluorophores in the compositions of the invention are usually quenched by the modified nucleoside in a detection compound, where the increase in fluorescence following cleavage of the polyphosphate linker is at least about 1.5 fold, at least about 2 fold, at least about 3 fold, at least about 4 fold, at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 200 fold, at least about 300 fold, at least about 400 fold, at least about 500 fold, at least about 600 fold, at least about 700 fold, at least about 800 fold, at least about 900 fold, at least about 1000 fold, at least about 2000 fold, at least about 3000 fold, at least about 4000 fold, or at least about 5000 fold.

### Enzymes and Detection Compounds

Nucleotide Pool Repair Enzymes.

The pool of nucleotides present in a cellular pool can be damaged in various ways, e.g. by reactive oxygen species. Nucleotides that are damaged, e.g. by oxidation, deamination, etc. are removed from the pool by specialized enzymes. Such enzymes include phosphohydrolases that act on modified nucleotides; and glycosylases that cleave damaged bases from ribose or deoxyribose. The compositions and method of the present invention are primarily directed to phosphohydrolases, which include without limitation, enzymes in the EC categories EC 3.6.1.55, EC 3.6.1.23, EC 3.1.3.56. Enzymes of interest in these classes include, without limitation, MTH1, dUTPase, and ITPase, and related enzymes in the NUDT family.

MTH1, also referred to as Nudix (Nucleoside Diphosphate Linked Moiety X)-Type Motif 1 (NUDT1), EC 3.6.1.55, hydrolyzes oxidized purine nucleoside triphosphates, such as 8-oxo-dGTP, 8-oxo-dATP, 2-hydroxy-dATP, and 2-hydroxy rATP, to monophosphates, thereby preventing misincorporation. Compounds for detection of MTH1 follow general formula I:

N-L-D I

where N is 8-oxoguanosine, 2′-deoxy 8-oxoguanosine, 2-hydroxyadenosine, deoxy 2-hydroxyadenosine; L is a polyphosphate of from about 3 to about 5 phosphates in length; and D is adenosine, or a fluorophore quenched by the nucleoside. Suitable fluorophores can be empirically tested for appropriate quenching. In some embodiments the fluorophore is pyrene. In some embodiments the fluorophore is 1,3-diaza-2-oxophenothiazine or the nucleoside thereof.

Exemplary detection compounds for MTH1 include, without limitation the following structures, any of which can be provided as an oxoguanosine, deoxyoxoguanosine, 2-hydroxyadenosine or deoxy 2-hydroxyadenosine (cleavage site marked by dashed line in A):

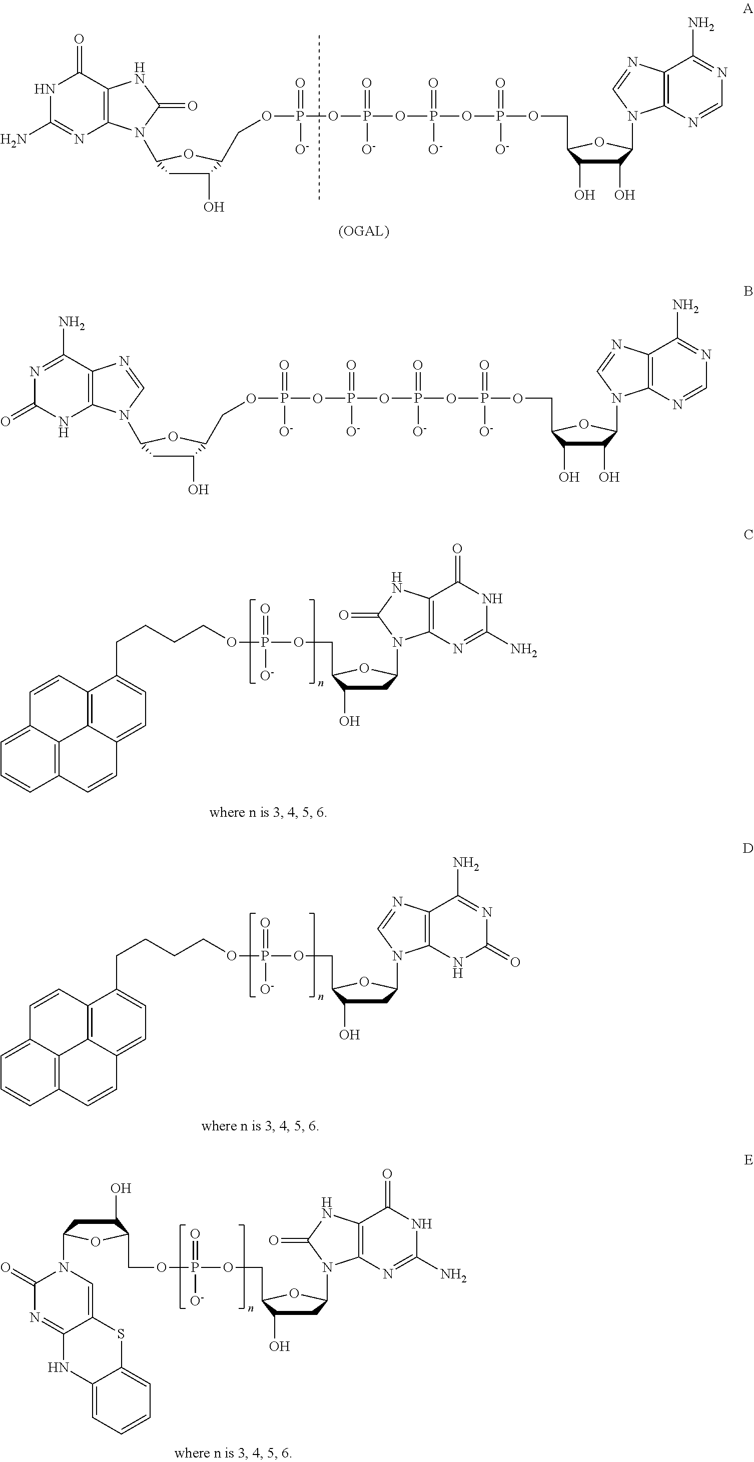

dUTPase, also referred to as DUTP pyrophosphatase, or DUT, EC 3.6.1.23 hydrolyzes dUTP to dUMP and pyrophosphate, thereby limiting intracellular pools of dUTP. Elevated levels of dUTP lead to increased incorporation of uracil into DNA, which induces extensive excision repair mediated by uracil glycosylase. This repair process can lead to DNA fragmentation and cell death.

dUTPase is important in therapeutic strategies for treating cancer by targeting thymidylate synthase (TS) pathway. Inhibitors of this pathway include the widely utilized fluoropyrimidine and antifolate classes of anti-cancer agents. Inhibition of TS induces a metabolic blockade, resulting in depletion of thymidylate and a corresponding accumulation of the TS substrate dUMP, which can be phosphorylated to dUTP. When cellular levels of dUTP increase, the activity of the enzyme that catalyses the reversion of this nucleotide to dUMP, dUTPase, can become saturated resulting and an excess of dUTP and misincorporation of uracil into DNA. In tumors with elevated expression of dUTPase, saturation of this enzyme activity is unlikely to occur and dUTP misincorporation is prevented as a mechanism of cytotoxicity. Metabolic conversion of 5-FU (and its prodrugs) to FdUTP and subsequent incorporation into DNA has also been shown to induce significant cytotoxicity and dUTPase has been shown to possess affinity for FdUTP, catalysing its hydrolysis to FdUMP and, therefore, preventing the misincorporation of FdUTP into DNA. In this context, specific dUTPase inhibitors can enhance the effectiveness of TS inhibition by promoting the accumulation and misincorporation of dUTP into DNA; by increasing the incorporation of FdUTP (when combined with fluoropyrimidine therapy) into DNA; and by prevent the recycling of dUTP. Thus measurement of dUTPase activity is clinically relevant.

Compounds for detection of dUTPase follow general formula I:

N-L-D I

where N is deoxyuridine; L is a polyphosphate of from about 3 to about 5 phosphates in length; and D is adenosine, or a fluorophore quenched by the deoxyuridine. Suitable fluorophores can be empirically tested for appropriate quenching. In some embodiments the fluorophore is pyrene.

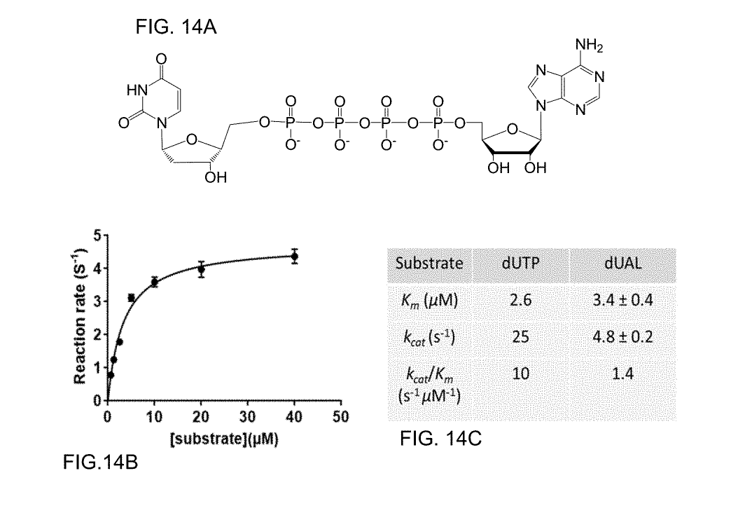

Exemplary detection compounds for dUTPase include, without limitation the following structures. Also see the exemplary DUAL probe shown in FIG. 14A.

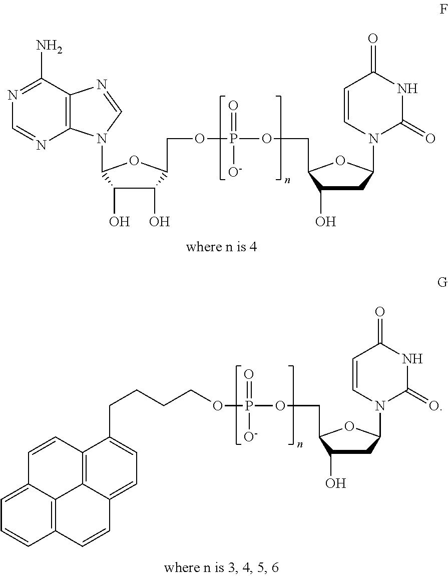

Inosine triphosphate pyrophosphatase (ITPA), EC 3.6.1.19, hydrolyzes deaminated purine nucleoside triphosphates, including ITP, dITP, XTP and dXTP, to their corresponding nucleoside monophosphate and pyrophosphate. ITPA is clinically important because a widespread polymorphism, 94C>A, leads to null ITPase activity in erythrocytes and is associated with an adverse reaction to thiopurine drugs. Individuals with defective ITPase are predisposed to genome damage by impurities in nucleotide pools, which is drastically augmented by therapy with purine analogs. They are also at an elevated risk for degenerative diseases and cancer. In some embodiments of the invention, a cellular sample from an individual is tested for levels of ITPase activity, where individuals with reduced activity relative to a normal control are disqualified from treatment with purine analogs, e.g. for treatment of cancer with thiopurine and related drugs.

Compounds for detection of ITPA follow general formula I:

N-L-D I

where N is inosine, deoxyinosine, xanthosine, deoxyxanthosine; L is a polyphosphate linker of from about 3 to about 5 phosphates in length; and D is adenosine, or a fluorophore quenched by the purine. Suitable fluorophores can be empirically tested for appropriate quenching. In some embodiments the fluorophore is pyrene.

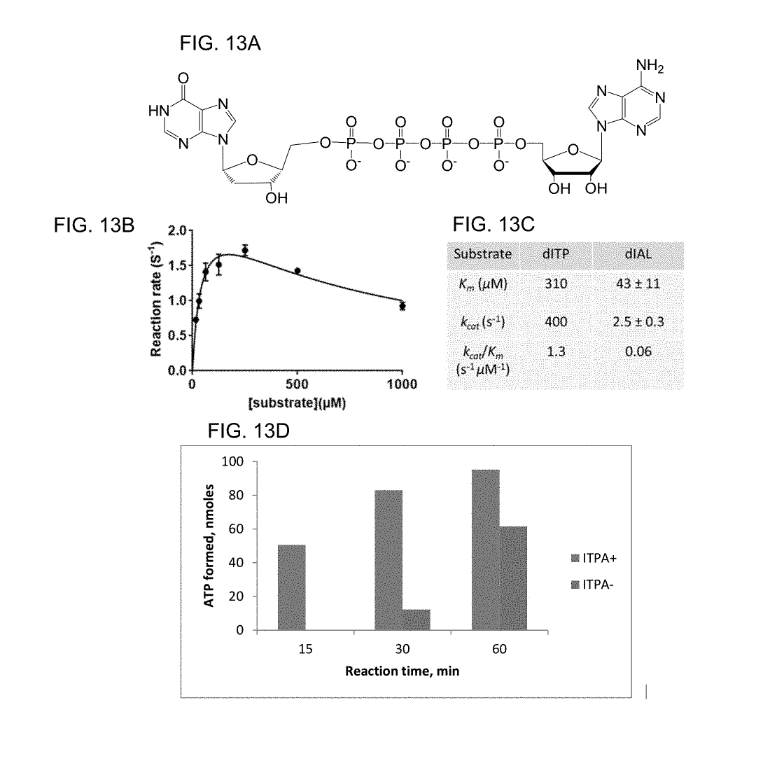

Exemplary detection compounds for ITPA include, without limitation the following structures (where inosine may replace deoxyinosine; and where xanthosine may replace deoxyxanthosine). Also see the exemplary DIAL probe shown in FIG. 13A.

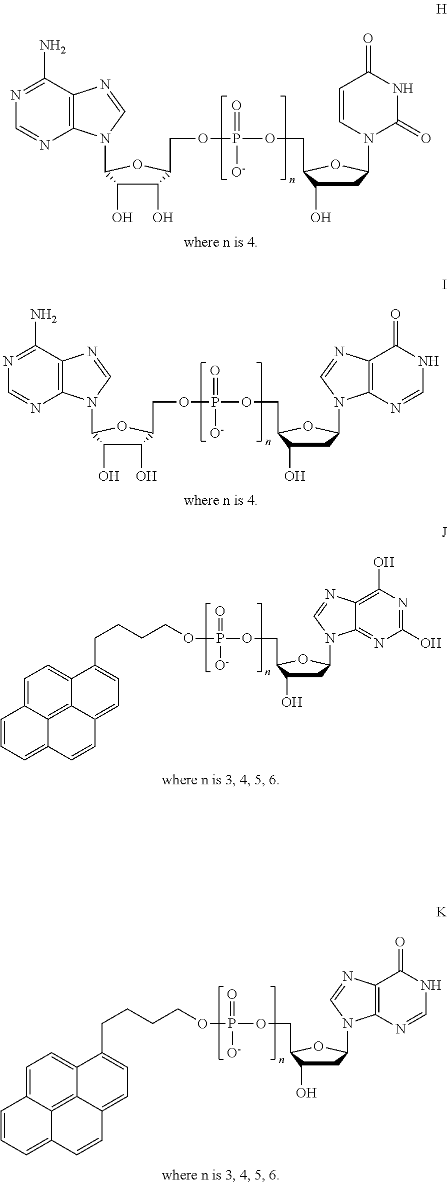

The compounds of the invention are provided in various forms appropriate for use, including solid, semi-solid, liquid or gaseous forms, such as tablets, capsules, powders, granules, solutions, gels, microspheres; as acids or salts, including physiologically acceptable salts. Excipients and buffers, such as vehicles, adjuvants, carriers or diluents, pH adjusting and buffering agents, tonicity adjusting agents, stabilizers, wetting agents and the like are commercially available. Useful salts retain the biological effectiveness and properties of the free acids, and are not biologically or otherwise undesirable. These salts can be prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, tertiary and quaternary amines, such as tetrabutylammonium; substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

### Methods

Activity of a nucleotide pool repair enzyme or enzymes is detected by combining detection compound of the invention, which have a cleavable bond that is susceptible to cleavage by the enzyme, with a test sample suspected of comprising the enzyme of interest. Where the enzyme is present, the polyphosphate linker L is cleaved, releasing detectable moiety. In some embodiments ATP is released as the detectable moiety. In other embodiments a fluorophore is cleaved from the quencher, and the resulting change in fluorescence is detected by any convenient method, as known in the art. Reference samples of known enzyme compositions may be included in an assay, or used to establish reference curves. Typically positive and negative controls are included. Enzymes of interest for analysis include any nucleotide pool repair enzyme with the capability of specifically acting on the modified nucleoside to cleave the polyphosphate linker between the substrate and the detectable moiety. Human enzymes are of particular interest, but the methods are not limited to such.

Assays using a detection compound where ATP is released provide a change in signal that results from the presence of released ATP, e.g. a fluorescent signal, light, etc. is generated. The signal is monitored and quantified with detectors, such as fluorescence spectrophotometers, microplate readers, UV lamps, PCR, commercial systems that allow the monitoring of fluorescence in real time reactions, or, in some instances, by the human eye. Where the detectable signal is light, e.g. from a luciferase based system, a wide range of lumimometer devices are commercially available for tubes, plates, multimodal plates, etc.

Samples comprising or suspected of comprising an enzyme of interest are conveniently added in solution, or readily soluble form to the detection compound, which may be provided in solution or bound to a substrate, such as a plate, well, bead, fiber, etc.

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. For ATP based assays, the sample is depleted of ATP prior to analysis. The term encompasses a clinical sample, and also includes cells in cell culture, cell supernatants, cell lysates, serum, plasma, biological fluids, and tissue samples. Also included are manufacturing and drug screening samples.

A plurality of assays may be run in parallel with different sample concentrations to obtain a differential response to the various concentrations. As known in the art, determining the effective concentration of an analyte typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the analyte or at or below the concentration of analyte that does not give a detectable change in fluorescence or ATP release.

Such assays may be conducted with samples obtained from a biological system under different environmental conditions, such as exposures to varying concentration of a drug candidate or mixtures of drug candidates, which can provide data on the efficacy, the safety profile, the mechanism of action and other properties of the drug candidates that are required in drug development. Alternatively, tissue samples may be probed for the presence of clinical conditions, e.g. for prognosis of cancer patients to treatment with an MTH1 inhibitor, for disqualification of individuals for treatment with thiopurine analogs; and the like.

In some embodiments, the methods of the invention are used in determining the efficacy of a therapy for treatment of a disease including cancer, e.g. cancers in which a ras oncogene is activated, either at an individual level, or in the analysis of a group of patients, e.g. in a clinical trial format. Such embodiments typically involve the comparison of two time points for a patient or group of patients. The patient status is expected to differ between the two time points as the result of a therapeutic agent, therapeutic regimen, or disease challenge to a patient undergoing treatment.

Examples of formats for such embodiments may include, without limitation, testing enzyme activity at two or more time points, where a first time point is a diagnosed but untreated patient; and a second or additional time point(s) is a patient treated with a candidate therapeutic agent or regimen.

In such clinical trial formats, each set of time points may correspond to a single patient, to a patient group, e.g. a cohort group, or to a mixture of individual and group data. Additional control data may also be included in such clinical trial formats, e.g. a placebo group, a disease-free group, and the like, as are known in the art. Formats of interest include crossover studies, randomized, double-blind, placebo-controlled, parallel group trial is also capable of testing drug efficacy, and the like. See, for example, Clinical Trials: A Methodologic Perspective Second Edition, S. Piantadosi, Wiley-Interscience; 2005, ISBN-13: 978-0471727811; and Design and Analysis of Clinical Trials: Concepts and Methodologies, S. Chow and J. Liu, Wiley-Interscience; 2003; ISBN-13: 978-0471249856, each herein specifically incorporated by reference.

Specific clinical trials of interest include analysis of therapeutic agents for inhibition of MTH1, where a patient is identified as having a cancer with upregulated MTH1 expression. Inhibitors for which clinical trials may be performed include, without limitation, (S)-crizotinib, TH287 and TH588, among others. Treatment may be performed in combination with additional chemotherapy, immunotherapy and/or radiotherapy. Patient responses to treatment may also be measured by the methods and compositions of the invention.

In one embodiment, a blinded crossover clinical trial format is utilized. A patient alternates for a set period of time, e.g. one week, two weeks, three weeks, or from around about 7-14 days, or around about 10 days, between a test drug and placebo, with a 4-8 week washout period. In another embodiment a randomized, double-blind, placebo-controlled, parallel group trial is used to test drug efficacy.

### Kits

Also provided are kits for practicing the subject methods. The kits according to the present invention may comprise at least one detection compound suitable for use in the methods described herein. A kit may further include reagents for detecting ATP, including, but not limited to one or both of: (a) an ATP-responsive fluorescent dye; (b) a luciferase and luciferin. A kit may further include additional reagents employed in the methods of the invention, e.g., buffers, nuclease inhibitors, polymerase inhibitors, etc. In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

The various reagent components of the kits may be present in separate containers, or may all be precombined into a reagent mixture for combination with samples. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

### Example 1

### A Chimeric ATP-Linked Nucleotide Enables Luminescence Signaling of Damage Repair by MTH1, a Cancer Target

The enzyme MTH1 cleanses the cellular nucleotide pool of oxidatively damaged 8-oxo-dGTP, preventing mutagenesis by this nucleotide. The enzyme is considered a promising new cancer therapeutic target; however, methods to measure its activity are indirect, laborious, and have low sensitivity. Here we describe the development of a novel chimeric nucleotide (OGAL) that enables luminescence signaling of the enzymatic reaction, greatly simplifying the measurement of MTH1 activity in vitro, in cell lysates, and in tumor tissue. Experiments confirm that the signaling reaction can be performed in one tube, yielding signals in minutes. We show that the reporting system can be used to identify and study inhibitors of MTH1, and we use it to quantify enzyme activity in eight cell lines and in colorectal tumor tissue. The OGAL reporter has considerable utility in the study of the biology of MTH1, and analysis of patient samples for diagnosis, prognosis, and during clinical testing of targeted therapeutics.

Cellular reactive oxygen species (ROS) can damage DNA in multiple ways. This includes not only the direct damage to the heterocyclic bases and deoxyribose sugars of DNA itself, but also to the cellular nucleotides that are used to synthesize new DNA during cell division. The most abundant forms of damage in the cellular nucleotide pool are 8-oxo-dGTP and 2-hydroxydATP; (Mo et al. Proc. Natl. Acad. Sci. U.S.A 1992, 89, 11021-11025; Maki et al. Nature 1992, 355, 273-275; Hah et al. Proc. Natl. Acad. Sci. U.S.A 2007, 104, 11203-11208) 8-oxo-dGTP is particularly dangerous in the cell because DNA polymerases can misincorporate it into DNA opposite A rather than C, leading to mutations (Pavlov et al. Biochemistry 1994, 33, 4695-4701; Kamiya Mutat. Res. 2010, 703, 32-36). To ameliorate this hazard, human cells express the enzyme MTH1, an 18 kDa homolog of bacterial MutT (Sakumi, et al. J. Biol. Chem. 1993, 268, 23524-23530; Hayakawa et al. Biochemistry 1995, 34, 89-95). Like its bacterial relative, human MTH1 binds 8-oxo-dGTP and hydrolyzes its triphosphate moiety between the α and β phosphates, producing inactive 8-oxo-dGMP and pyrophosphate. Thus the enzyme is critically important for cleansing the nucleotide pool of damage that can cause cellular mutations.

Significantly, while this activity is important for suppressing mutations in normal cells, it is not essential for cell viability (Tsuzuki et al. Proc. Natl. Acad. Sci. U.S.A 2001, 98, 11456-11461). However, cancer cells can be highly dependent on MTH1 to maintain their rapid growth (Giribaldi et al. Oncotarget 2015, 6, 11519-11529). Many tumors are driven by mutations in the RAS proto-oncogenes that increase reactive oxygen species, resulting in damage such as 8-oxo-dG. Thus RAS-dependent tumor cells often express high MTH1 levels to counteract the toxicity of elevated ROS in these rapidly growing cells (Patel et al. Oncogene 2015, 34, 2586-2596; Coskun et al. DNA Repair 2015, 33, 101-110).

Such studies suggest the promise of targeting MTH1 as a new therapeutic approach for multiple RAS-driven cancers such as melanoma and colorectal cancer (Gad et al. Nature 2014, 508, 215-221; Huber et al. Nature 2014, 508, 222-227). Thus the inhibition of this enzyme is quite promising from the clinical oncology perspective. In developing targeted therapies for MTH1, it will therefore be important to measure enzyme activity in tumor specimens, for patient selection, to monitor responses to therapy, and to evaluate any resistance that arises. Moreover, simple high-throughput methods for quantifying MTH1 activity are valuable for identifying new inhibitors and improving existing ones.

Despite the growing clinical significance of this enzyme, current methods to measure MTH1 are laborious and indirect, and have low sensitivity. The most common methods include HPLC measurement of nucleotide cleavage to 8-oxo-dGMP, which is limited by the relatively low UV absorbance of the nucleotide, and a colorimetric assay involving the use of pyrophosphatase to degrade the pyrophosphate byproduct to inorganic phosphate, which is then detected after complexation with molybdate and malachite green dye. (See Takagi et al. J. Biol. Chem. 2012, 287, 21541-21549; Feng et al. Anal. Biochem. 2011, 409, 144-149) Because of the low sensitivity and laborious nature of these methods, most studies do not measure MTH1 activity, but instead evaluate indirectly related metrics such as mRNA quantities or protein levels, which may not correlate linearly with cellular enzyme activity. A recent study, citing the need for measuring MTH1 for clinical development, reported a proteomics method involving isotope labeling, protease digestion, HPLC and tandem mass spectrometry, which due to its complexity may be difficult to develop for routine clinical use. In addition, for most applications, it would be preferable to measure MTH1 activity rather than protein quantities, since the enzyme may have variable activity due to single nucleotide variations (Goto et al. J Genet. 2008, 87, 181-186).

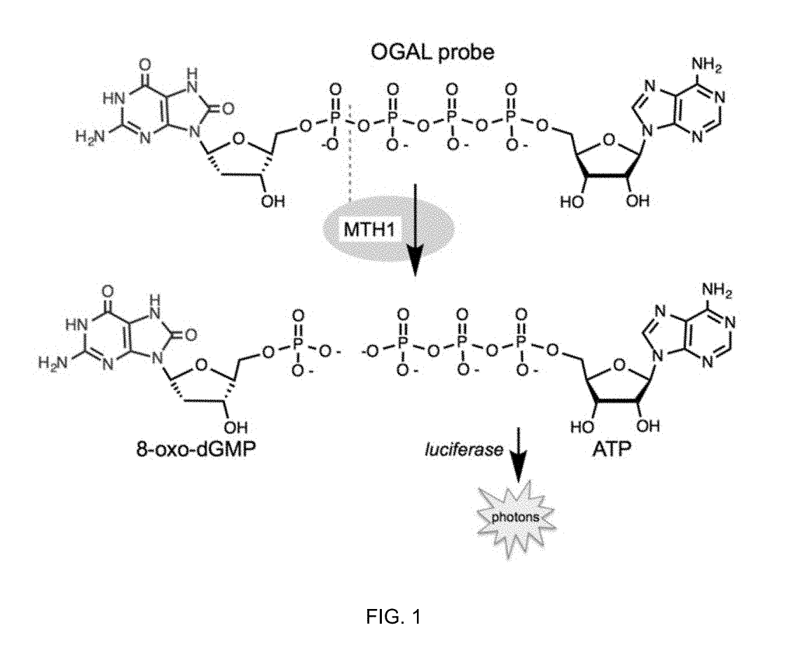

Here we report the development of a simple chimeric MTH1 substrate that enables luminescence or fluorescence reporting of this enzyme's activity. When the enzyme acts on this two-headed dinucleotide (an oxo-G ATP-linked (OGAL) probe (FIG. 1)), ATP is released, generating a luminescence signal with luciferase. This allows the rapid and sensitive measurements of enzyme activity in vitro, in cell lysates, and in tumor tissue specimens.

Our design of a specific reporter for MTH1 began with the knowledge that MTH1 performs hydrolytic cleavage between the α and β phosphates of 8-oxo-dGTP, releasing pyrophosphate. Examination of the structure of MTH1 with product bound shows that the terminus of the leaving pyrophosphate group is likely exposed to solution, thus allowing its exit upon bond cleavage. By appending adenosine monophosphate at this position instead (FIG. 1), the leaving group in this reaction is adenosine 5′-triphosphate (ATP), which is widely employed with luciferase to generate luminescent signals. Unknowns in this design included (a) whether the AMP conjugation with 8-oxo-dGTP would hinder MTH1 activity, and (b) whether luciferase might (in an undesired outcome) accept the chimeric nucleotide as a substrate, thus short-circuiting the design and preventing the use of the reporter for MTH1.

Synthesis of the OGAL probe was achieved by converting ATP and 8-oxo-dGMP sodium salts to tetrabutylammonium salts, and then activating the ATP with carbonyl diimidazole and reacting it with the 8-oxo-dGMP. The resulting chimeric dinucleotide was purified by reverse phase HPLC, giving 45% yield, and was characterized by NMR and mass spectrometry.

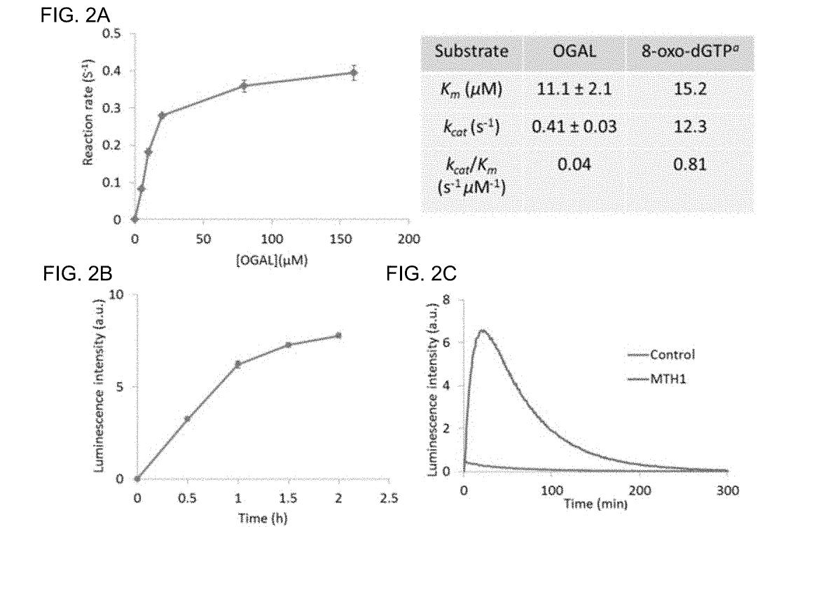

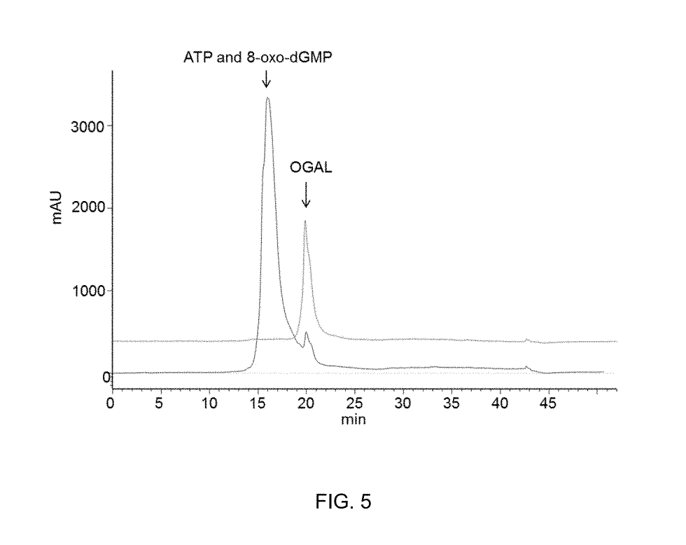

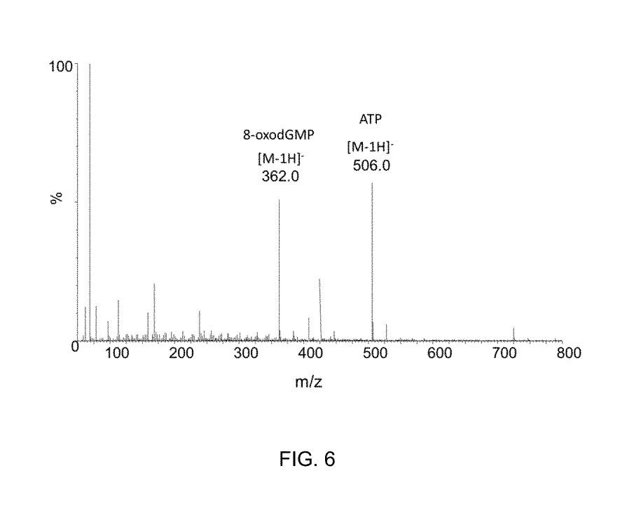

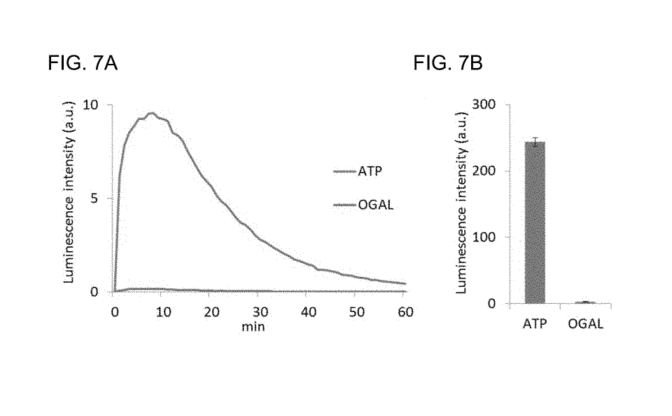

We proceeded to characterize the responses of the probe to MTH1 and luciferase. Incubation of the probe with MTH1 cleanly consumed the OGAL probe and produced an overlapping peak containing two products, which coelute with ATP and 8-oxo-dGMP by analytical HPLC (FIG. 5). Mass spectrometry confirmed these products (FIG. 6). We carried out kinetics studies of the OGAL substrate with MTH1 (FIG. 2), which revealed a Kₘ of 11.1±2.1 μM, within experimental error of the reported value of the native substrate, and a k_{cat} of 0.41±0.03 s⁻¹, which is lower than that of the native 8-oxo-dGTP by a factor of ca. 30. Next we incubated the OGAL substrate with luciferase and monitored signal as compared with native ATP; results showed only very small signals for the OGAL dinucleotide (FIG. 7). The signals appear rapidly and then drop to zero, suggesting that they arise from small amounts of contaminating ATP in the synthetic nucleotide, rather than from a slow reaction of OGAL with luciferase.

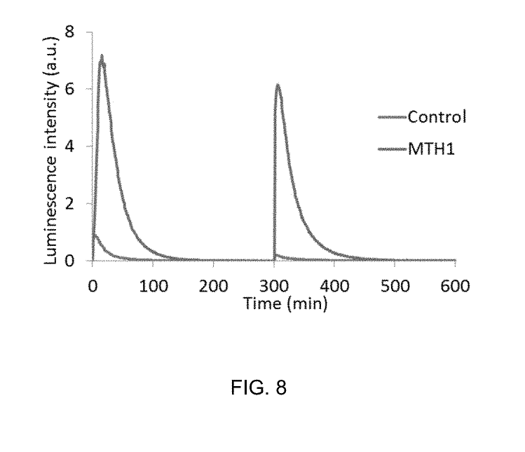

Next we explored strategies for coupling OGAL/MTH1 reaction with luciferase reporting. To avoid possible buffer incompatibilities and interferences, we first tested a two-tube method. We performed a 100 μL MTH1 reaction with 40 μM OGAL, removing 5 μL aliquots over time, and using the aliquots to generate luciferase signals in a commercial luciferase buffer, reading the luminescence signal in a microplate luminometer at the standard 5 min luciferase reaction time. A plot of the data reveals signals rising over background within 5 min and reaching a plateau in ca. 120 min of MTH1 reaction time (FIG. 2B). This two-tube approach offers some convenience in analyzing multiple samples accumulated over time, and for cell lysate measurements (see below). However, in many applications it would be more convenient if the MTH1 activity could be measured in a single reaction. Thus we tested a single tube approach, adding OGAL probe and luciferase in luciferase reaction buffer, and then measuring luminescence after MTH1 is added. A time plot of luminescence (FIG. 2C) shows signal increasing over an hour, then decreasing as expected with luciferase signaling. Subsequent experiments adding a second aliquot of luciferase reaction buffer confirmed that the drop in signal is from eventual loss of activity of luciferase rather than from MTH1 enzyme instability in luciferase buffer (FIG. 8).

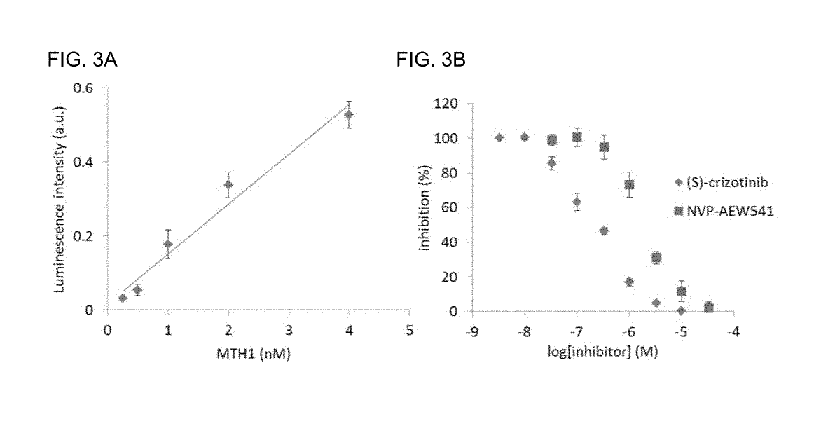

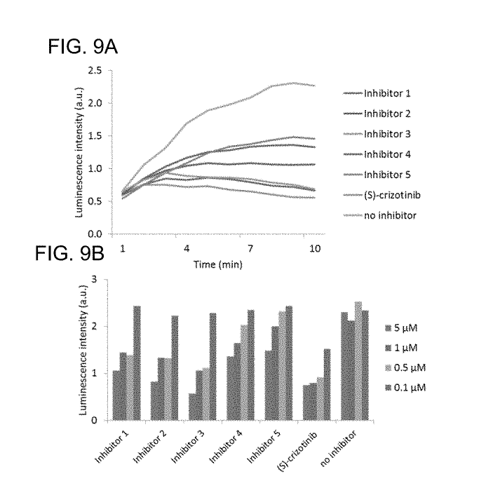

We evaluated the sensitivity of the OGAL reporter by varying MTH1 concentration and measuring subsequent luciferase signals. A plot of the data (FIG. 3) shows a limit of detection of 0.5 nM enzyme concentration. Later experiments (see below) showed that this sensitivity is sufficient to measure native levels of MTH1 in multiple tumor cell lysates. Because of the clinical interest in targeting MTH1, we confirmed the ability of the OGAL reporter to characterize small-molecule inhibitors of this enzyme. (S)-Crizotinib was recently reported as an MTH1 inhibitor with a reported IC₅₀ of 72 nM and 500 nM as measured by inorganic pyrophosphate assay and malachite green assay respectively. We performed inhibitor dilution experiments in the presence of OGAL and MTH1; plots of reaction rate vs. time (FIG. 3B) revealed an IC₅₀ of 220 nM. To test whether the new probe could be used in library screening format to identify new inhibitors, we applied the one-tube method to evaluate a set of known kinase inhibitors, which led to the identification of NVP-AEW541 (Gorre et al. Science 2001, 293, 876-880), previously unknown as an inhibitor of MTH1 (FIG. 9). Measurement of an inhibition curve (FIG. 3B) for this new compound revealed an apparent IC₅₀ of 1.9 μM. Thus the OGAL assay provides a convenient method for screening libraries and identifying previously unknown inhibitors of MTH1.

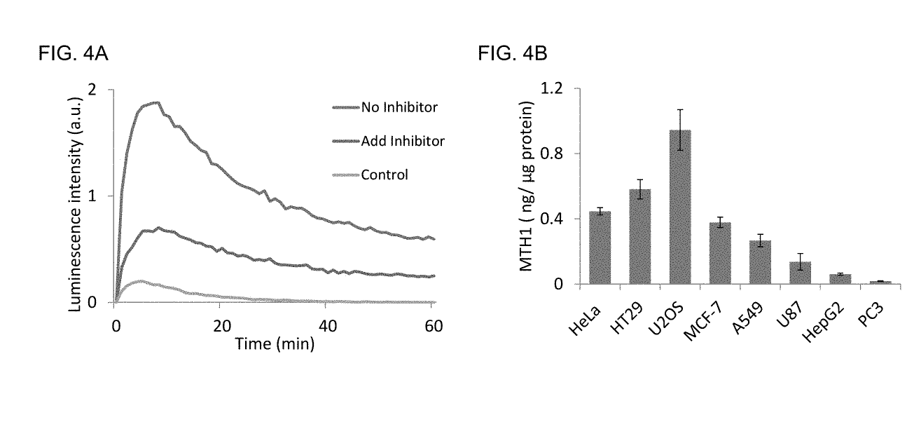

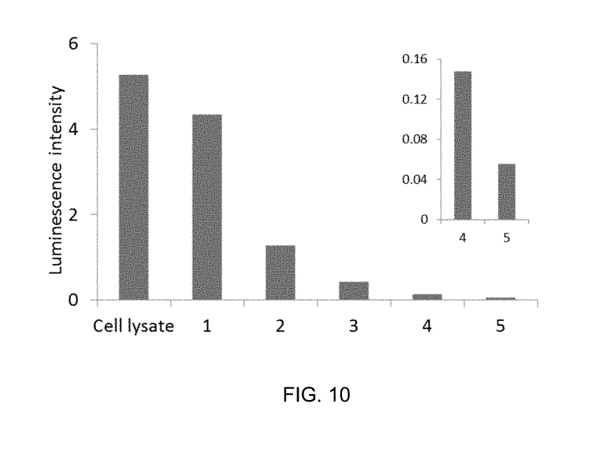

Beyond the in vitro use of the OGAL/luciferase system for evaluating MTH1 enzyme and inhibitors, it would be useful if this molecular probe could measure MTH1 activities from cells, since levels of this enzyme are reported to be upregulated in RAS-dependent tumor cells and tissues. Thus we carried out experiments of the probe with lysates of tumor cell lines. Because cells contain millimolar quantities of ATP, we used a spin filtration column to deplete lysates of ATP prior to assay; experiments with U2OS cell lysate confirmed near-complete removal of constitutive ATP signals after five washes (FIG. 10). The remaining lysate fraction was re-diluted in MTH1 reaction buffer for measurement of cellular activity. A time course with 40 μM OGAL (FIG. 4) revealed substantial luminescence signals from 1-2 μg of cellular total protein. We measured MTH1-specific signals by performing the same experiment in the presence of 20 μM inhibitor (S)-crizotinib, which reduced signals by 70%. Since our in vitro studies showed that this concentration of the inhibitor gives essentially complete MTH1 inhibition (FIG. 3B), we assign the remaining signal to background from other cellular enzyme activities. Quantitative subtraction of this background signal from a given cell lysate then allows a measure of MTH1 activity in that lysate.

We proceeded to quantify MTH1 activities in a variety of tumor cell lines, several of which have not been previously characterized for this enzyme activity. Enzyme activities were quantified in protein mass terms by interpolation using our previous dilution curve for purified enzyme (FIG. 3). Results showed (FIG. 4B) that MTH1 activities varied widely (by a factor of 45) in the cell lines; U2OS cells have the highest levels of those measured, while PC3 cells have the lowest. Three of these cell lines (HeLa, MCF-7 and HepG2) were previously measured for MTH1 levels via mass spectrometry methods. Those results reported that HeLa and MCF-7 cells have similarly high levels of protein, the same as the result observed here. In addition, the ratio of levels of MTH1 previously measured in HeLa vs. HepG2 cells (6:1 respectively) is also very close to that observed here (7:1), providing independent confirmation of our approach. We note that our method is considerably simpler and less costly, and measures activity directly rather than protein amount.

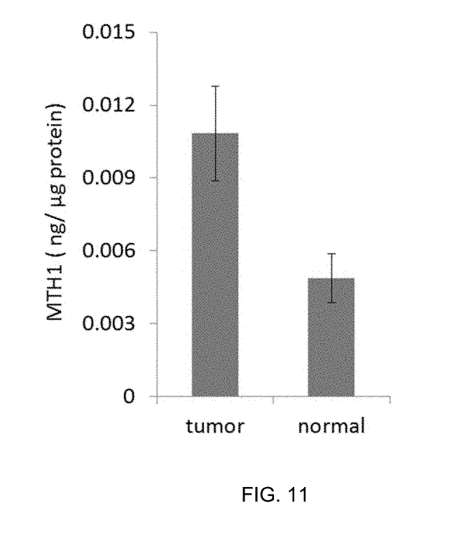

Given the ability of the OGAL reporter to quantify MTH1 activity from cultured cells, we next carried out a preliminary test of its capacity to report on the enzyme in a primary human tumor specimen. We obtained frozen RAS mutation-positive tumor tissue and normal colon tissue from patient with colorectal cancer, and prepared ATP-depleted lysates from it. We then used the OGAL reporter to measure a tumor MTH1 activity of 0.011±0.003 ng/μg cellular protein (FIG. 11). Comparison to a normal colon tissue sample revealed an MTH1 level of 0.005±0.003 ng/μg, thereby establishing a 2.2-fold elevated signal in the tumor (p<0.01). These values fall within the range reported recently for tumor specimens as measured by the prior proteomics/mass spectrometry method. Thus we conclude that the OGAL probe methodology can be used to assess MTH1 status in a clinically relevant cancer sample by simple and rapid luminescence measurements.

In sum, we have shown that the OGAL probe design functions effectively to signal MTH1 activity in vitro and in cell and tissue lysates. No other luminescence method exists for measuring this enzyme's activity. The new method is rapid and simple, and can be used to quantitate activity directly rather than indirect metrics such as mRNA or protein amount. Compared to an earlier malachite green (MG) assay, the OGAL sensitivity is greater due to the high sensitivity of luciferase for ATP. Moreover, the MG assay is not suitable for cell lysates, while the current method is the only existing approach shown to measure activity from cells. The enzymatic efficiency is relatively high for the OGAL probe; while the reduced k_{cat} value slows the generation of signals relative to the theoretical maximum of the native substrate, we still observe maximal signals in conveniently short amounts of time (30-120 min). More importantly, the Kₘ value of the OGAL substrate is identical to that of the native damaged nucleotide, which should allow for identification of new inhibitors.

The OGAL probe can enable the quantitative selection of patients for MTH1-targeted therapy by analysis of biopsy specimens. Since the probe measures activity directly rather than protein quantity, it can be used to report on differential activity of mutants as well.

### Experimental Methods

Instrumentation and Chemicals.

¹H and ³¹P NMR spectra were recorded on a Varian Mercury 400 MHz NMR spectrometer. ¹H and ³¹P NMR spectra were internally referenced to the residual solvent signal. Semi-preparative high performance liquid chromatography was performed on a LC-20AD Shimadzu liquid chromatography system, equipped with a SPD-M20A diode array detector and a CBM-20A system controller and using reverse phase (C18) columns. Absorbance spectra were obtained on a Cary 100 Bio UV-Vis spectrometer. The bioluminescence signal was recorded by microplate fluorometer (Fluoroskan Ascent, Thermal).

Chemicals were purchased from commercial suppliers (Sigma-Aldrich or Alfa Aesar) unless otherwise indicated. 8-Oxo-2′-deoxyguanosine was purchased from Berry & Associates. Human MTH1 full length protein purchased from Abcam (Cambridge, USA). ATP Determination Kit (A22066) from Life Technologies (Invitrogen) was used for the bioluminescence assay. MTH1 inhibitor (S)-Crizotinib was purchased from Sigma-Aldrich. A Bio-Rad Protein Assay Kit was used for determination of the protein concentration of cell lysates. The Kinase inhibitor Screening Library purchased from Cayman Chemical (Ann Arbor, Mich., USA)

OGAL Probe: Synthesis and Purification.

8-Oxo-2′-deoxyguanosine was phosphorylated as previously reported (Yoshikaw et al. Tetrahedron Left. 1967, 8, 5065-5068) to give the corresponding monophosphates (8-oxo-dGMP). The ammonium salt of 8-oxo-dGMP was obtained in 80% yield after purification by DEAF Sephadex G-25 anion exchange column. The ammonium salt of 8-oxo-dGMP and sodium salt of ATP were dissolved in distilled deionized water and converted into their free acids using a Dowex-50W ion exchange column (H⁺ form), titrated to pH 7.0 with a dilute solution of tetrabutylammonium hydroxide, and then lyophilized twice to powder. The lyophilized powder of the nucleotides were coevaporated with anhydrous DMF twice and kept under high vacuum for 3 h before the subsequent coupling reaction (below).

The tetrabutylammonium salt of ATP (160 mg, 100 μmol) was dissolved in 1.5 mL anhydrous DMF. To the solution, carbonyldiimidazole (CDI, 100 mg, 600 μmol) was added, and the mixture was stirred at room temperature for 6 h, after which 150 μL MeOH was added to quench the reaction. All solvents were removed under high vacuum and the residue redissolved in 1 mL anhydrous DMF. The 8-oxo-2′-deoxyguanosine monophosphate tetrabutylammonium salt (60 mg, 60 μmol) in 1 mL DMF and anhydrous MgCl₂ (25 mg) were added. The mixture was stirred for 72 h at room temperature. After this, the product was precipitated by the addition of acetone (20 mL). The precipitate was washed twice with 10 mL acetone. The desired product was purified by reverse-phase HPLC (RPHPLC) using a preparative C18 column with a gradient of acetonitrile and 50 mM triethylammonium acetate buffer (pH 7). Fractions containing pure product were concentrated and further purified by a DEAF Sephadex G-25 anion exchange column, eluted with 500 mM NH₄HCO₃. The fractions containing the desired product were pooled, concentrated and repeatedly freeze-dried to yield the final product as a white powder in a yield of 45%. ¹H NMR (D₂O, 400 MHz, NH₄⁺ form): δ 8.30 (s, 1H), 7.98 (s, 1H), 5.93-5.88 (m, 2H), 4.54-4.51 (m, 2H), 4.38-4.36 (m, 1H), 4.20-4.18 (m, 1H), 4.12-4.06 (m, 3H), 3.94-3.91 (m, 2H), 2.87-2.83 (m, 1H), 2.00-1.98 (m, 1H). ³¹P NMR (D₂O, 162 MHz, NH₄⁺ form): δ-10.32 (m, 2P), −22.38 (m, 2P). HRMS: calculated for C₂₀H₂₉N₁₀O₂₀P₄ (M+H)+853.0505, found 835.0503.

OGAL Probe Assay.

Two-tube OGAL probe reaction: A standard 20 μL reaction buffer contained: 50 mM NaCl, 10 mM Tris-HCl (pH 7.9), 10 mM MgCl₂, 1 mM DTT, 40 μM OGAL nucleotides, and varies amount of MTH1. After 1 h incubation at 30° C., 5 μL of this reaction solution was added to 95 μL luciferase reaction solution (prepared as instructed for the ATP determination kit) in a 96 well plate. The bioluminescence signal was recorded at 1 min intervals over 1 h by microplate fluorometer.

One-tube OGAL probe reaction: 40 μM (final concentration) OGAL probe was added in 100 μL luciferase reaction buffer, and incubated at 30° C. for 1 h to eliminate the small amount of ATP contaminant in OGAL. After that, 10 nM MTH1 was added and the luminescence signals were recorded for 5 h at 1 min intervals.

Michaelis-Menten Analysis of MTH1 Using OGAL Probe.

A Michaelis-Menten curve was generated by adding MTH1 (20 nM) in 50 mM NaCl, 10 mM Tris-HCl (pH 7.9), 10 mM MgCl₂, 1 mM DTT and various amount of OGAL (5-160 μM) at 30° C. for 5 min. Then take 5 μL added to 95 μL luciferase reaction solution to calculate the amount of ATP generated by MTH1 reaction. All the reactions were performed in three replicates. The velocity was plotted as a function of OGAL concentration and fit with the Michaelis-Menten equation to obtain the kinetic parameters, Vₘₐₓ and Kₘ. The k_{cat} values were calculated by dividing the Vₘₐₓ by the concentration of MTH1 used.

Inhibitor Screening and IC₅₀ Determination Methods.

The screening of inhibitors employed the one tube OGAL probe assay reaction. The kinase inhibitors diluted from 10 mM to 100 μM in a 96 wells plate. 5 μL 100 μM inhibitor was mixed with 5 μL 400 nM MTH1 and incubated at 30° C. for 5 min in 96 wells plate, then added into 90 μL luciferase reaction buffer containing 20 μM OGAL. The luminescence signals were recorded for 1 h at 1 min intervals. The luminescence signals at 10 min were used to monitor the inhibition activity. The positive inhibitors were rescreened with luciferase reaction buffer containing 1 μM ATP (no OGAL probe or MTH1) to rule out any inhibitors of luciferase.

Inhibitory concentration (IC50) values were determined by the two-tube OGAL probe assay. The reaction buffer contained: 50 mM NaCl, 10 mM Tris-HCl (pH 7.9), 10 mM MgCl₂, 1 mM DTT, 40 μM OGAL nucleotides, and varied concentrations of inhibitors. After 15 min incubation at 30° C., 5 μL of this reaction solution was added to 95 μL luciferase reaction solution in a 96 well plate to measure ATP signals. All the reactions were performed as three replicates. The IC₅₀ was calculated using GraphPad Prism software.

Preparation of ATP-depleted cell lysates. PC3 cells (ATCC) and A549 cells (ATCC) were grown in F-12K Medium (Kaighn's Modification of Ham's F-12 Medium, ATCC), HT-29 (ATCC) and U2OS cells (ATCC) were grown in McCoy's 5A Medium, and HeLa (ATCC), HepG2 (ATCC), MCF7 (ATCC) and U87 MG (ATCC) cells were grown in ATCC-formulated Eagle's Minimum Essential Medium. All media was supplemented with 10% fetal bovine serum (FBS), 100 U/ml of penicillin and 100 μg/ml of streptomycin, and cells were grown in a humidified incubator at 37° C. with 5% CO₂. 6×150 mm plates of cells were grown to 90% confluence. The preparation of cell lysate adapted from the published method (Folco et al. J. Vis. Exp. 2012, e4140). Cells (about 10⁷ cells) were was harvested and washed twice with PBS buffer, then the cells were swelled in hypotonic buffer (10 mM HEPES, pH 7.9, 1.5 mM MgCl₂, 10 mM KCl, 0.5 mM DTT, 1× protease inhibitor cocktail with 1 mM Na₃VO₄ as polymerase inhibitor). The cells were incubated on ice for 30 min with vortexing every 10 min. Cells were further disrupted and homogenized cells by passing through a 16 gauge needle for 10-15 times. The crude lysates were centrifuged at 13000 rpm for 10 min at 4° C. The supernatant is the whole cell lysate. To deplete ATP in these cell lysates, the supernatant was transferred to Amicon 3K centrifugal filter device (EMD Millipore) and centrifuged at 13000 rpm for 10 min at 4° C. The filtrate was discarded, and the concentrate was reconstituted to original sample volume with hypotonic buffer. This step was repeated 5 times to completely remove the ATP, and this yielding about 100 μL ATP-depleted cell lysate. The cell lysate was transferred to pre-chilled tubes and kept at −80° C. until use. Protein concentrations in these cell lysates were measured by Bio-Rad Protein Assay Kit (Bradford method).

Preparation of Colon Tissue Lysates.

Colon tumor and normal tissue were from Stanford Tissue Bank. The colon tumor tissue (>60% tumor) and normal tissue were from a male patient who was diagnosed with invasive adenocarcinoma with the KRAS G13D and BRAF V600E mutations.

The tissues (stored at −80° C.) were cut into small pieces quickly. Approximately 20 mg of the tissue was transferred to a 1.5 mL Eppendorf tube, and 500 μL hypotonic buffer (10 mM HEPES, pH 7.9, 1.5 mM MgCl₂, 10 mM KCl, 0.5 mM DTT, 1× protease inhibitor cocktail with 1 mM Na₃VO₄ as polymerase inhibitor) was added. The tissue was homogenized thoroughly using a handheld homogenizer and the sample kept on ice for 30 min with occasional vortexing. The crude lysates were centrifuged at 13000 rpm for 10 min at 4° C. The supernatant was retained as the whole lysate. The ATP depletion from these tissue lysates was then performed by the same method as the cultured cell lysates (above). The protein concentrations were measured by Bio-Rad Protein Assay Kit.

Quantifying MTH1 in Cell and Tissue Lysates.

Cell lysates were diluted to 1 μg/μL; 2 μL of each diluted cell lysate was added to 20 μL MTH1 reaction buffer with or without 20 μM (S)-crizotinib. The MTH1 reaction buffer contained 50 mM NaCl, 10 mM Tris-HCl (pH 7.9), 10 mM MgCl₂, 1 mM Na₃VO₄, 1 mM DTT, 40 μM OGAL nucleotide and 0 μM or 20 μM (S)-crizotinib. After 1 h incubation at 30° C., 5 μL of this reaction solution was added to 95 μL luciferase reaction solution in a 96 well plate, and the luminescence signal at 5 min was recorded. To calculate MTH1-specific signals, background level with inhibitor for each lysate was subtracted from that without inhibitor. To obtain absolute enzyme levels, these data were fit to the standard dilution plot of signal from pure MTH1 (see FIG. 3A, main text). All data were the averages from three replicates. For the detection of MTH1 activity in tissue lysate, we use the same method as detection MTH1 in cancer cell lysate, except with 30 μg total protein in each reaction. The OGAL luminescence signals in tumor and normal colon samples was measured 9 times for each lysate.

Note that our titrations of (S)-crizotinib with pure full-length MTH1 show complete inhibition at 10 μM (FIG. 3B). Thus we use the lysate signal with inhibitor as the baseline signal with no MTH1 activity. We attribute the remaining background signal in ATP-depleted cells from other unknown enzyme activities on the OGAL probe. This approach is validated by results that give good agreement with literature values in cultured cell lines and tissues.

### Example 2

### Measurement of ITPA Activity

### General Procedure for Measuring Inosine Triphosphaphate Pyrophosphatase (ITPA) Activity in Vitro

DIAL probe assay. Two-tube DIAL probe reaction: A standard 20 μL reaction buffer contained: 50 mM Tris-HCl 50 mM MgCl2 1 mM DTT pH 8.5, 20 μM DIAL nucleotides (FIG. 13A), and varies amount of ITPA. After 1 h incubation at 37° C., 5 μL of this reaction solution was added to 95 μL luciferase reaction solution (prepared as instructed for the ATP determination kit) in a 96 well plate. The bioluminescence signal was recorded at 1 min intervals over 1 h by microplate fluorimeter, shown in FIG. 13B.

Michaelis-Menten Analysis of ITPA Using DIAL Probe.

A Michaelis-Menten curve was generated by adding ITPA (20 nM) 50 mM Tris-HCl 50 mM MgCl₂ 1 mM DTT pH 8.5, and various amount of DUAL (16-100 μM) at 37° C. for 10 min. Then take 5 μL added to 95 μL luciferase reaction solution to calculate the amount of ATP generated by ITPA reaction. All the reactions were performed in three replicates. The velocity was plotted as a function of DIAL concentration and fit with the Michaelis-Menten equation to obtain the kinetic parameters, Vₘₐₓ and Kₘ. The k_{cat} values were calculated by dividing the Vₘₐₓ by the concentration of ITPA used. Shown in FIG. 13C.

Measuring ITPA activity in E. coli extracts. We used substrate (DIAL probe)—100 μM final concentration, reaction time 15 or 30 min, t=37° C., protein concentration=50 ng/μL. The data show that the activity can be clearly measured in the bacterial extract, whereas bacteria lacking the ITPA expression show no detectable signal.

ATP formed, nM

| Strain | 15 min | 30 min |
|---|---|---|
| wt | 1206.3 ± 70.3 | 1360.5 ± 63 |
| rdgB− | Not Detected | Not Detected |

Measuring ITPA activity in yeast extracts with DIAL probe. We used 100 μM DIAL probe and crude yeast cells extract (0.5 μg of total protein per 10 μL reaction). Reaction time varied over 15 min-1 hour, temperature was 30° C. The data show that ITPA activity can be measured readily in yeast expressing the gene, whereas at early (15 min) time, no measurable signal was detected in cells lacking this gene expression. At longer times (1 hr), background signal was seen from the ITPA probe, suggesting some cross reactivity of other enzymes with the DIAL probe. However, at short times the ITPA-specific signal was clear. Shown in FIG. 13D.

### Example 3

### Enzymatic Data for DUAL Probe

DUAL probe assay. Two-tube dUAL probe reaction: A standard 20 μL reaction buffer contained: 70 mM Tris-HCl 10 mM MgCl2 5 mM DTT pH 7.6 20 μM DUAL nucleotides (structure shown in FIG. 14A), and varies amount of MTH1. After 1 h incubation at 30° C., 5 μL of this reaction solution was added to 95 μL luciferase reaction solution (prepared as instructed for the ATP determination kit) in a 96 well plate. The bioluminescence signal was recorded at 1 min intervals over 1 h by microplate fluorometer. Shown in FIG. 14B.

Michaelis-Menten analysis of dUTPase using dUAL probe. A Michaelis-Menten curve was generated by adding dUTPase (2 nM) 70 mM Tris-HCl 10 mM MgCl2 5 mM DTT pH 7.6 and various amount of DUAL (1-40 μM) at 30° C. for 3 min. Then take 5 μL added to 95 μL luciferase reaction solution to calculate the amount of ATP generated by dUTPase reaction. All the reactions were performed in three replicates. The velocity was plotted as a function of dUAL concentration and fit with the Michaelis-Menten equation to obtain the kinetic parameters, Vmax and Km. The kcat values were calculated by dividing the Vmax by the concentration of dUTPase used. Shown in FIG. 14C.

1. A detection compound having the structure:
N-L-D I
where N is a modified nucleoside substrate for a nucleotide pool repair enzyme;
L is a polyphosphate linker of from about 3 to about 6 phosphates in length; and
D is a detectable moiety selected from (i) adenosine and (ii) a fluorophore that is quenched when linked to the modified nucleoside, but detectable when released.

2. The detection compound of claim 1, wherein the modified nucleoside is selected from oxoguanosine, deoxyoxoguanosine, 2-hydroxyadenosine, deoxy 2-hydroxyadenosine, deoxyuridine, inosine, deoxyinosine, xanthosine, and deoxyxanthosine.

3. The detection compound of claim 1, wherein the detectable moiety is adenosine and the polyphosphate linker is 4 phosphates in length.

4. The detection compound of claim 1, wherein the detectable moiety is a fluorophore and the increase in fluorescence following cleavage of the linker is at least about 10-fold.

5. The detection compound of claim 1 wherein the detectable moiety is pyrene or an analog thereof.

6. The detection compound of claim 1 wherein the detectable moiety is a tricyclic cytosine analog or nucleoside analog thereof.

7. The detection compound of claim 6, wherein the detectable moiety is 1,3-diaza-2-oxophenothiazine.

8. The detection compound of claim 1, having a structure selected from

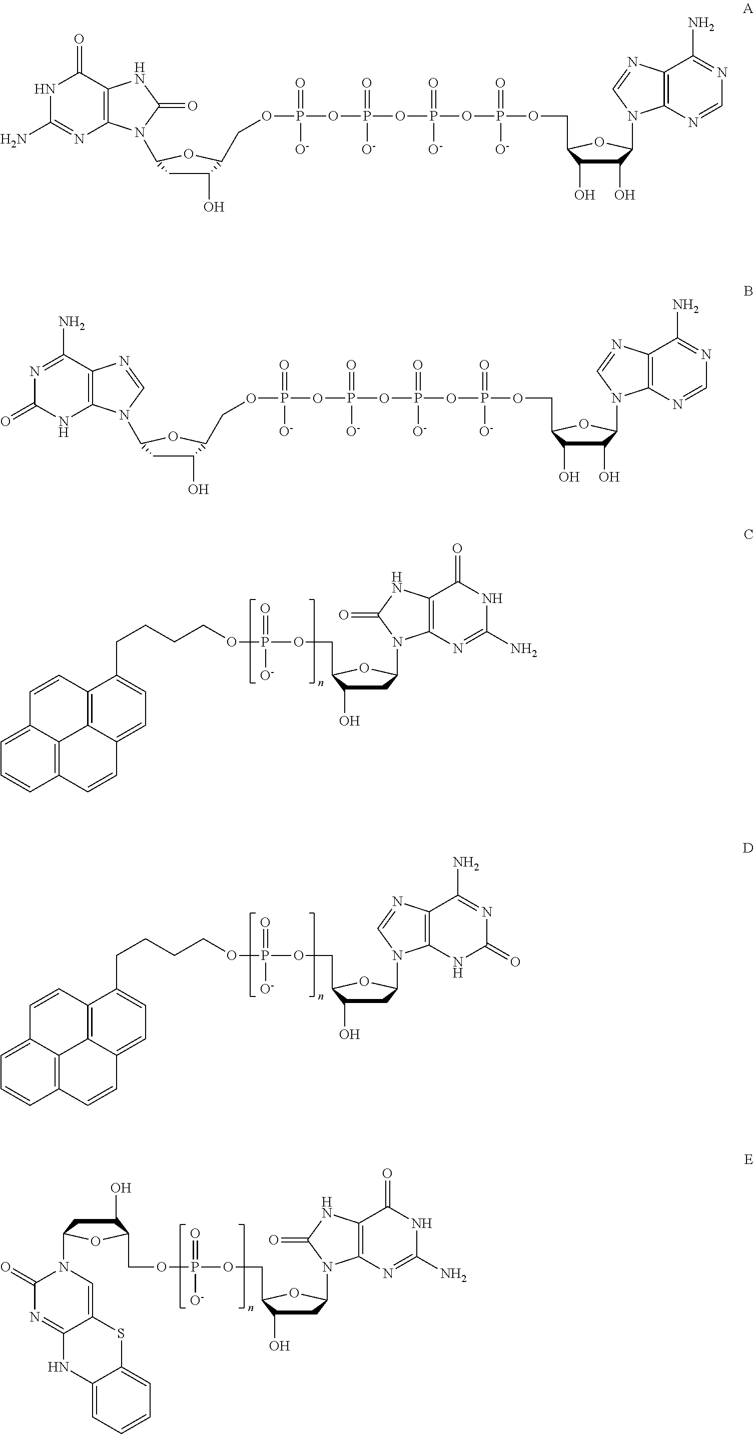

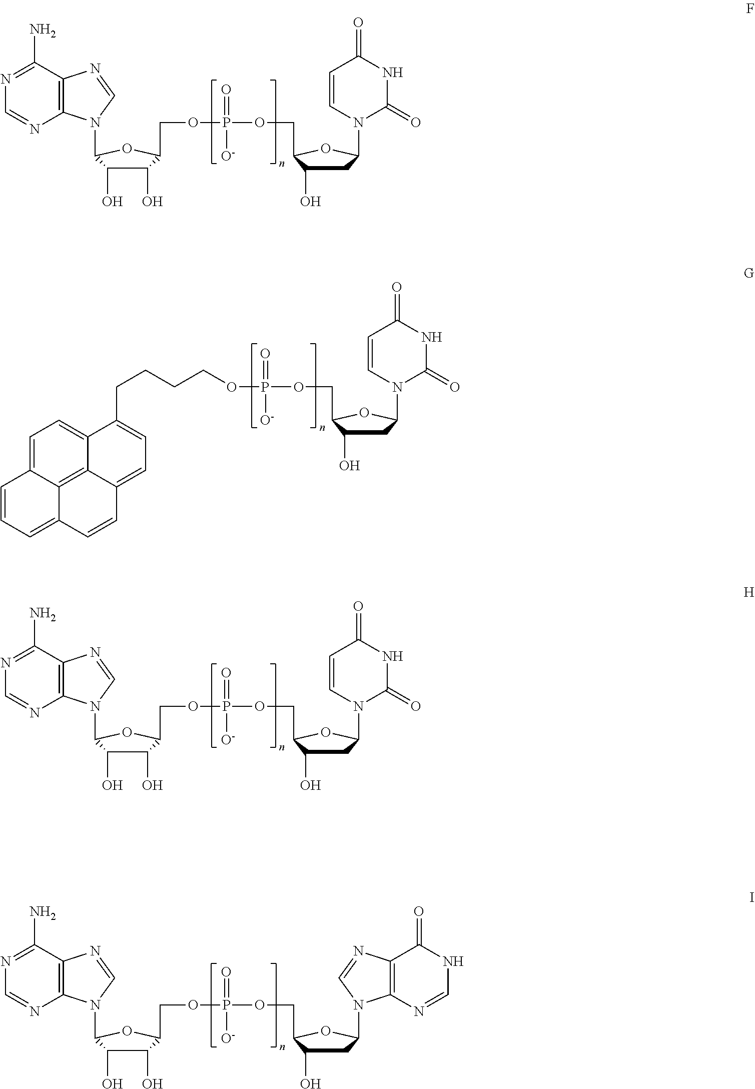

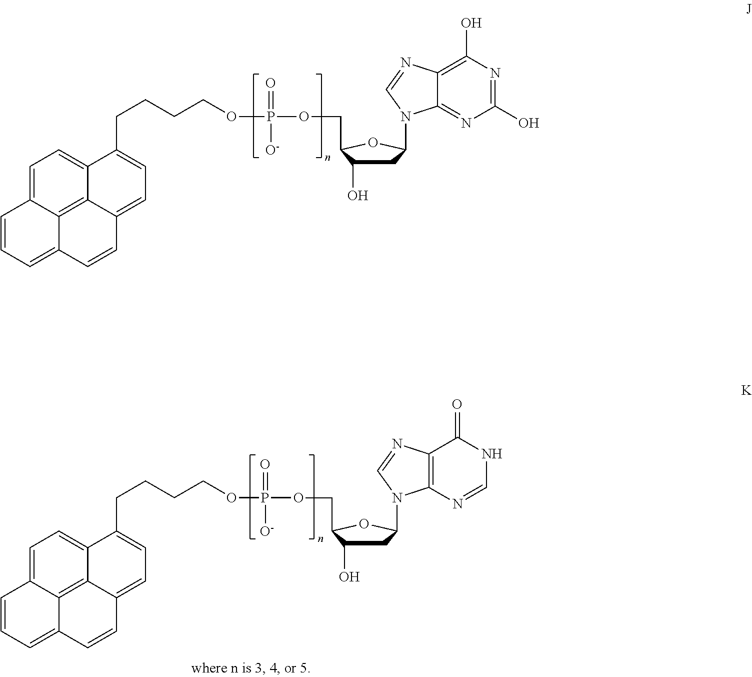

9. A method for detection of a nucleotide pool sanitization enzyme of interest, the method comprising:
contacting a sample suspected of comprising said nucleotide sanitization enzyme of interest with the detection compound of any one of claims 1-8 under conditions that permit cleavage of the linker by the enzyme; and
measuring the released detectable moiety.

10. The method of claim 9, wherein the nucleotide pool sanitization enzyme is one or more of MTH1, dUTPase, and ITPase.

11. The method of claim 9, wherein the released detectable moiety is measured by detection of increased fluorescence.

12. The method of claim 9, wherein released ATP is measured by contacting the reaction mixture with luciferin and an ATP-dependent luciferase enzyme to produce light.

13. The method of claim 9, wherein released ATP is measured by contacting the reaction mixture with an ATP-responsive fluorescent dye.

14. The method of claim 12, wherein the sample is depleted of ATP prior to contacting with the detection compound.

15. The method of claim 9, wherein the sample comprises a drug candidate for inhibition or potentiation of nucleotide pool repair activity.

16. The method of claim 9, wherein the sample is a biological sample.

17. The method of claim 16, wherein the biological sample is a cell or cell lysate from a patient.

18. The method of claim 17, wherein the patient is a cancer patient.

19. A kit for detection of nucleotide pool sanitization enzyme activity, comprising:
a detection compound of claim 1; and
instructions for use.

20. The kit of claim 19, further comprising luciferin and luciferase.
